(19) United States
(12) Patent Application Publication
(10) Pub. No.: US 2020/0291341 A1
(43) Pub. Date: Sep. 17, 2020

(54) PSEUDO SAMPLE CREATION METHOD AND APPARATUS, AND NUMERICAL MODEL CREATION SYSTEM AND METHOD

(71) Applicant: HITACHI, LTD., Tokyo (JP)

(72) Inventors: Shunsuke KONO, Tokyo (JP); Takuya KAMBAYASHI, Tokyo (JP); Toshimitsu NOGUCHI, Tokyo (JP); Akihiro NOJIMA, Tokyo (JP)

(73) Assignee: HITACHI, LTD., Tokyo (JP)

(21) Appl. No.: 16/774,048

(22) Filed: Jan. 28, 2020

(30) Foreign Application Priority Data
Mar. 12, 2019 (JP) 2019-044420

(51) Int. Cl. C12M 1/06; C12N 5/071; C12M 1/34

(52) U.S. Cl. CPC C12M 27/02; C12M 41/00; C12N 5/0682

(57) ABSTRACT

Human labor and time can be reduced, and a high-accuracy numerical model can be created. Provided is a pseudo sample creation method including mixing a pre-culture liquid medium that is not used to culture a sample, a post-culture liquid medium that is used to culture the sample, and an additive containing at least one of saccharides, an amino acid, a metabolite, and protein with one another to create a pseudo sample. In addition, provided is a numerical model creation system including: a spectrometer that acquires spectral data of a pseudo sample; a reference value measuring device that measures a reference value of the pseudo sample; and an analyzer that creates a numerical model representing a correlation between the spectral data and the reference value based on the spectral data and the reference values obtained from a plurality of pseudo samples.

### TECHNICAL FIELD

The present invention relates to a pseudo sample creation method, a pseudo sample creation apparatus, a numerical model creation system, and a numerical model creation method.

### BACKGROUND ART

As a method of grasping the progress status after culturing a sample such as cells, microorganisms, or fungi in a culture solution, a method of measuring a predetermined component (for example, a component derived from the sample or a component as a nutrient of the sample) in the culture solution by optical analysis is known. In the optical analysis, the culture solution including the sample is non-destructively, non-invasively, and instantaneously analyzable. Accordingly, the culture solution after the optical analysis is reusable, that is, culture of a sample in the culture solution after the optical analysis can be further carried out. Therefore, the optical analysis is widely used to grasp the progress status of culturing a sample using a culture solution. In order to measure a predetermined component in a culture solution using optical analysis, it is necessary to create a numerical model in advance.

The numerical model is a model representing a correlation between spectral data obtained by optical analysis and the concentration of a predetermined component in a culture solution.

In the related art, in order to create a numerical model, it is necessary to carryout the process of actually culturing a sample using a culture solution multiple times. That is, for the culture, a variation in the concentration of the above-described predetermined component is generated during multiple times of the culture processes. Therefore, by using data obtained during multiple times of the culture processes for creating a numerical model, a high-accuracy numerical model can be constructed in consideration of a variation in the concentration of a predetermined component in a culture solution.

As described above, in the related art, it is necessary to perform culture processes multiple times to create a numerical model, and massive human labor and time is required.

In order to reduce the human labor and time, for example, NPL 1 describes a method of creating a numerical model after adding glucose or the like as a material to be measured to a culture solution of sampled cells to increase the signal intensity of the material to be measured for the numerical model.

### CITATION LIST

### Non-Patent Literature

NPL 1: Jiang Qiu, et al., “On-line near infrared bioreactor monitoring of cell density and concentrations of glucose and lactate during insect cell cultivation”, Journal of Biotechnology 173, 106-111, 2014

### SUMMARY OF INVENTION

### Technical Problem

When the method described in NPL 1 is used for a culture solution in which the concentration of a component changes over time, it is necessary to carry out periodic sampling of a culture solution, mixing of a material to be measured, and optical measurement or the like multiple times. Therefore, human labor and time is still required.

The present invention has been made under these circumstances, and an object thereof is to reduce required human labor and time such that a high-accuracy numerical model can be created.

### Solution to Problem

The present application includes a plurality of means for solving the above-described problem, and one example thereof is as follows.

In order to solve the above-described problems, according to one aspect of the present invention, there is provided a pseudo sample creation method including mixing a pre-culture liquid medium that is not used to culture a sample, a post-culture liquid medium that is used to culture the sample, and an additive with one another to create a pseudo sample.

### Advantageous Effects of Invention

According to one aspect of the present invention, it is possible to reduce required human labor and time to create a high-accuracy numerical model.

Objects, configurations, and effects other than those described above will be clarified through the description of the following embodiments.

### BRIEF DESCRIPTION OF DRAWINGS

FIG. 1 is a diagram illustrating the summary of a pseudo sample creation method according to a first embodiment of the present invention.

FIG. 2 is a diagram illustrating the summary of the pseudo sample creation method according to the first embodiment of the invention.

FIG. 3 is a flowchart illustrating an example of the pseudo sample creation method.

FIG. 4 is a diagram illustrating a configuration example of a numerical model creation system according to a second embodiment of the invention.

FIG. 5 is a diagram illustrating a configuration example of a spectrometer.

FIG. 6 is a diagram illustrating an example of a shape of an analysis cell.

FIG. 7 is a diagram illustrating a configuration example of an analyzer.

FIG. 8 is a diagram illustrating an effect of a created pseudo sample.

FIG. 9 is a diagram illustrating a configuration example of a pseudo sample creation system according to a third embodiment of the invention.

FIG. 10 is a diagram illustrating a configuration example of a culturing device.

FIG. 11 is a diagram illustrating a configuration example of a mixing device.

FIG. 12 is a diagram illustrating a configuration example of an analysis cell introducing device.

### DESCRIPTION OF EMBODIMENTS

Hereinafter, a plurality of embodiments of the present invention will be described with reference to the drawings. In all diagrams for describing each of the embodiments, basically, the same members are represented by the same reference numerals, and the description thereof will not be repeated. In addition, in the following embodiments, it goes without saying that the components (including element steps and the like) are not necessarily required, unless expressly stated otherwise and unless they are considered to be clearly required in principle or other reasons. In addition, when referring to “including A”, “composed of A”, “having A”, and “containing A”, it goes without saying that the elements other than the specified one should not be excluded, unless expressly stated the fact that there is only the particular element, or other reasons. Also, in the following embodiment, when referring to the shape, the positional relationship, or other characteristics of the components and the like, elements that substantially approximate or similar to the shape or other characteristics are included unless expressly stated otherwise and unless they are clearly considered not to be so in principle.

### Summary of Pseudo Sample Creation Method According to First Embodiment of Present Invention

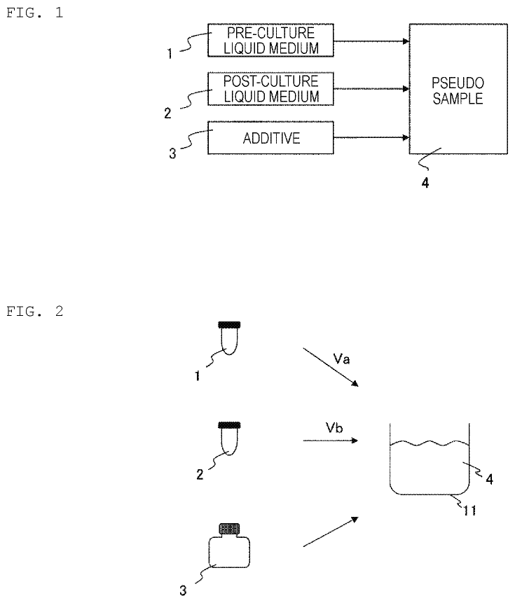

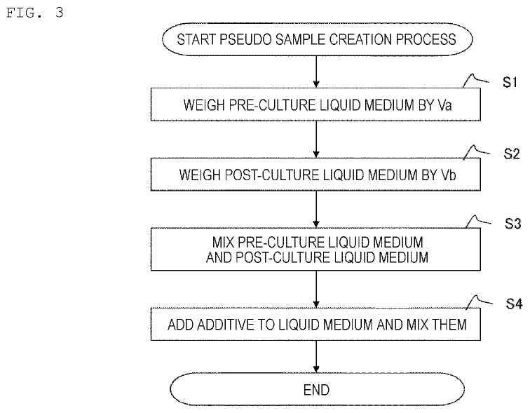

FIGS. 1 and 2 are diagrams illustrating the summary of a pseudo sample creation method according to a first embodiment of the present invention.

In the pseudo sample creation method according to the first embodiment of the invention, a pre-culture liquid medium 1, a post-culture liquid medium 2, and an additive 3 are mixed with one another to create a pseudo sample 4.

<Definitions of Pre-Culture Liquid Medium 1, Post-Culture Liquid Medium 2, and Additive 3>

The pre-culture liquid medium 1 is a liquid medium that is not used to culture a sample. The pre-culture liquid medium 1 does not contain saccharides and an amino acid. However, the pre-culture liquid medium 1 may contain a predetermined amount or less of at least one of saccharides and an amino acid.

The post-culture liquid medium 2 is a liquid medium that is obtained when culture of a sample such as cells, microorganisms, or fungi ends. The post-culture liquid medium 2 may remove the sample such as cells, microorganisms, or fungi from the liquid medium after culture by filtering or the like. Here, the time when the culture of the sample ends is the time when the concentration of a metabolite such as lactic acid produced by metabolic activity of the sample exceeds a predetermined threshold. The predetermined threshold is a value of the concentration of the metabolite in a state where the culture process is assumed to end and can be freely defined by a user.

The additive 3 contains at least one of saccharides, an amino acid, a metabolite, and protein. The additive 3 maybe in any form, for example, a solid such as powder or a tablet or a liquid such as a solution.

The pseudo sample 4 is a liquid that is created by mixing the pre-culture liquid medium 1, the post-culture liquid medium 2, and the additive 3.

<Tools Used to Create Pseudo Sample 4>

In order to create the pseudo sample 4, a pipette for weighing each of the pre-culture liquid medium 1, the post-culture liquid medium 2, and the additive 3, a stirring device such as a stirrer or a spatula for mixing the components, and a container for storing the pseudo sample 4 are used.

<Method of Creating Pseudo Sample 4>

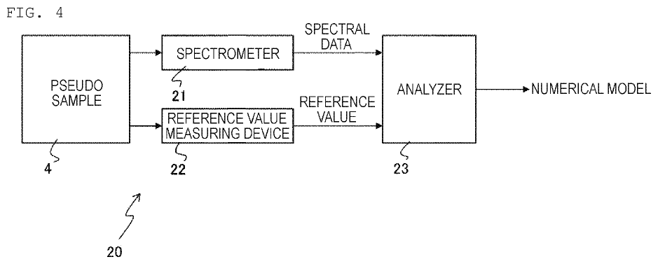

FIG. 3 is a flowchart illustrating an example of the method of creating the pseudo sample 4.

First, a creator weighs the pre-culture liquid medium 1 by Va [ml] using the pipette (Step S1). Next, the creator weighs the post-culture liquid medium 2 by Vb [ml] using the pipette (Step S2). Next, the creator stores and mixes the weighed pre-culture liquid medium 1 and the weighed post-culture liquid medium 2 with each other in the container (Step S3).

At this time, the liquid medium having a volume of Vc (=Va+Vb) [ml] is stored in the container. A proportion of each of the volume Va of the pre-culture liquid medium 1 and the volume Vb of the post-culture liquid medium 2 to the volume Vc may be a value in a range of 0% to 100% (including 0% and 100%).

Next, the creator creates the pseudo sample 4 by adding a predetermined amount of the additive 3 to the liquid medium in which the pre-culture liquid medium 1 and the post-culture liquid medium 2 are mixed with each other and sufficiently mixing the components with one another (Step S4). During mixing, the stirring device such as a stirrer or a spatula may be used, or the container maybe covered with a lid and shaken by the creator.

Using the above-described creation method, a plurality of pseudo samples 4 having different mixing ratios among the pre-culture liquid medium 1, the post-culture liquid medium 2, and the additive 3 are created.

When the additive 3 is insoluble, the additive 3 that remains without being dissolved may be removed from the mixed pseudo sample 4 using a filter or the like.

### Numerical Model Creation System 20 According to Second Embodiment of Present Invention

Next, FIG. 4 illustrates a configuration example of a numerical model creation system 20 according to a second embodiment of the present invention.

The numerical model creation system 20 includes a spectrometer 21, a reference value measuring device 22, and an analyzer 23.

The spectrometer 21 performs spectrometry of the pseudo sample 4 created by the creator and outputs spectral data obtained by the spectrometry to the analyzer 23.

The reference value measuring device 22 is an existing device adopting a method other than spectrometry such as an enzyme reaction or chromatography. The reference value measuring device 22 measures the concentration of a predetermined component in the pseudo sample 4 created by the creator and outputs the result to the analyzer 23 as a reference value.

The analyzer 23 creates a numerical model representing a correlation between the spectral data and the reference value by multi-variable analysis based on the spectral data and the reference values obtained from the plurality of pseudo samples 4.

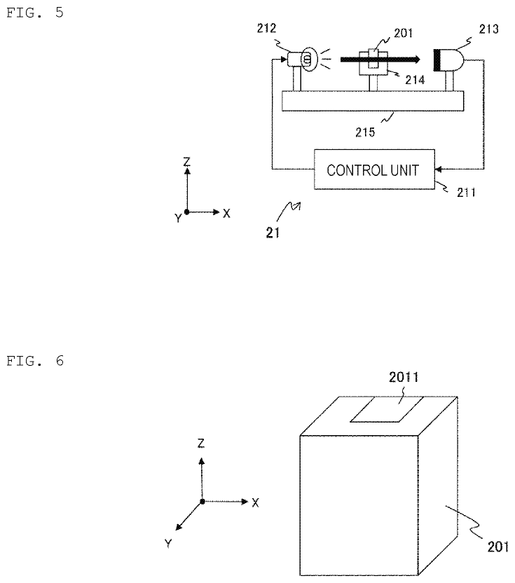

Next, FIG. 5 illustrates a configuration example of the spectrometer 21.

The spectrometer 21 includes a control unit 211, a light source unit 212, a light receiving unit 213, a holding unit 214, and a fixing tool 215. An XYZ coordinate system in the spectrometer 21 is as illustrated in the drawing, and the same is applied to the subsequent drawings.

The control unit 211 is configured with, for example, a computer including a CPU (Central Processing Unit), a memory, a storage, and a communication interface, and each function of the control unit 211 is implemented by the CPU executing a predetermined program. The control unit 211 controls emission of the light source unit 212 and creates spectral data based on a light receiving signal from the light receiving unit 213. The controller 211 may transmit the light receiving signal from the light receiving unit 213 to the analyzer 23 without creating the spectral data such that the analyzer 23 creates the spectral data. In addition, the control unit 211 may also function as the analyzer 23 described below.

For example, the light source unit 212 emits light having a predetermined wavelength (for example, infrared light, near infrared light, visible light, ultraviolet light, or X-ray) to the pseudo sample 4 that is stored in an analysis cell 201 held by the holding unit 214. The light receiving unit 213 is configured with, for example, a photomultiplier tube, a Si photodiode, an InGaAs photodiode, or a PbS photoconductive cell. The light receiving unit 213 receives transmitted light emitted from the light source unit 212 and transmitted through the pseudo sample 4, and outputs a light receiving signal representing a light receiving effect to the control unit 211. The light receiving unit 213 may be positioned where not only the transmitted light transmitted through the pseudo sample 4 but also light reflected or scattered from the pseudo sample 4 can be received.

The holding unit 214 is formed of, for example, a material such as plastic or metal that is not likely deformed. The holding unit 214 is held by and fixed to the analysis cell 201 in which the pseudo sample 4 is stored.

The fixing tool 215 maintains a relative positional relationship among the light source unit 212, the light receiving unit 213, and the holding unit 214.

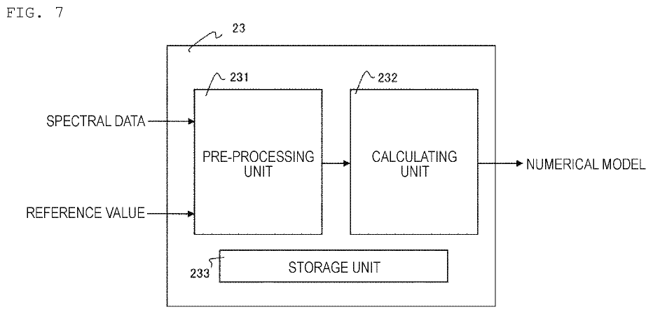

Next, FIG. 6 illustrates an example of a shape of the analysis cell 201.

The analysis cell 201 is a container for storing the pseudo sample 4, and is formed to have at least one pair of planes facing each other. In the same drawing, an external shape and an internal space of the analysis cell 201 are formed of a rectangular parallelepiped. The analysis cell 201 is formed of a material that allows transmission of light emitted from the light source unit 212, for example, fused silica or an acrylic resin.

On an upper surface of the analysis cell 201, an introduction port 2011 for introducing the pseudo sample 4 is provided. In addition, on a lower surface of the analysis cell 201, a discharge port (not illustrated) for discharging the pseudo sample 4 may be provided.

In addition, the entirety of the analysis cell 201 is put into a box or the like to block external light, and an opening for allowing the light emitted from the light source unit 212 to be incident into the box and an opening for allowing the transmitted light to exit may be provided.

The analysis cell 201 is reusable, and the pseudo sample 4 may be replaced multiple times for spectrometry. In addition, a plurality of the same analysis cells 201 may be prepared such that the analysis cell 201 to be used can be replaced at any timing. When the analysis cell 201 is reused, it is necessary to replace the pseudo sample 4. However, labor required to replace the analysis cell 201 can be reduced. When a plurality of the same analysis cells 201 are used, a decrease in analysis accuracy caused by carry-over can be prevented.

Next, an example of a spectral data creation process by the spectrometer 21 will be described.

In the spectrometer 21, background measurement is performed. In the background measurement, for example, a light receiving signal representing a state where the empty analysis cell 201 not storing the pseudo sample 4 is held by the holding unit 214 is acquired, or a light receiving signal representing a state where the analysis cell 201 is not held by the holding unit 214 is acquired. By subtracting the result of the background measurement from a light receiving signal representing a state where the analysis cell 201 storing the pseudo sample 4 is held by the holding unit 214, true spectral data of the pseudo sample 4 can be obtained.

First, a measurer (that may be the same as or different from the creator of the pseudo sample 4) cleans the analysis cell 201 using pure water, a cleaning solution (for example, an organic solvent), or the pseudo sample 4. When the analysis cell 201 is not used, cleaning is not necessarily performed. During cleaning, the cleaning solution or the like may be introduced from or may be discharged to the introduction port 2011 of the analysis cell 201.

After cleaning the analysis cell 201, the measurer introduces the pseudo sample 4 from the introduction port 2011 of the analysis cell 201. After the introduction, the analysis cell 201 is held by and fixed to the holding unit 214.

Next, the light source unit 212 starts emitting light having a predetermined wavelength to the analysis cell 201 storing the pseudo sample 4 in accordance with the control from the control unit 211. The light receiving unit 213 starts receiving light transmitted through the analysis cell 201 in accordance with a control signal from the control unit 211, and outputs a light receiving signal representing a light receiving effect to the control unit 211. The control unit 211 creates spectral data based on the light receiving signal. Next, the measurer disposes the pseudo sample 4 stored in the analysis cell 201 and ends a single spectrometry operation.

By repeating the above-described spectrometry by at least the number of the pseudo samples 4, the spectral data creation process by the spectrometer 21 ends.

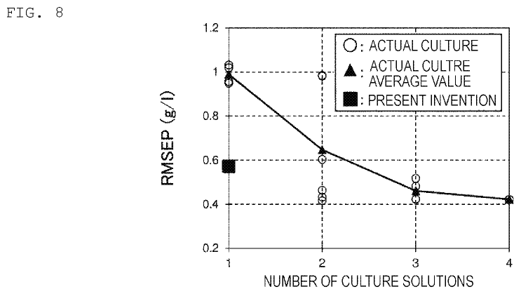

Next, FIG. 7 illustrates a configuration example of functional blocks configuring the analyzer 23.

As in the control unit 211 of the above-described spectrometer 21, the analyzer 23 is configured with a computer, and each of the functions of the analyzer 23 is implemented by a CPU executing a predetermined program. The analyzer 23 may also function as the control unit 211. The analyzer 23 includes a pre-processing unit 231, a calculating unit 232, and a storage unit 233.

The pre-processing unit 231 performs a predetermined pre-processing (for example, four arithmetic operation, differential, integral, or normalization) on the spectral data and the reference values obtained from the pseudo samples 4 in order to convert the spectral data and the reference value into a data format suitable for multi-variable analysis in the calculating unit 232 described below, and outputs the results to the calculating unit 232. The pre-processing unit 231 is not necessarily provided.

The calculating unit 232 creates a numerical model representing a correlation between the spectral data and the reference value by performing multi-variable analysis on the pre-processed spectral data and the pre-processed reference values obtained from the plurality of pseudo samples 4.

As a method of the multi-variable analysis, for example, single regression analysis, multiple regression analysis, quantification theory type I, quantification theory type II, quantification theory type III, discriminant analysis, logistic regression analysis, principal component analysis, partial least squares regression (PLS regression), factor analysis, cluster analysis, correspondence analysis, multidimensional scaling, conjoint analysis, support vector machine, decision tree, Random forest, naive bayes, neural network, or deep learning can be assumed. However, multi-variable analysis other than the above-described examples may be adopted.

The storage unit 233 is used as a work area, for examples, stores data in the process of calculation by the pre-processing unit 231 or the calculating unit 232.

Next, an example of a numerical model creation process by the analyzer 23 will be described.

In the analyzer 23, first, the pre-processing unit 231 performs a predetermined pre-processing on the spectral data obtained from the spectrometer 21 and the reference values obtained from the reference value measuring device 22, and outputs the results to the calculating unit 232. Next, the calculating unit 232 creates a numerical model by performing multi-variable analysis on the pre-treated spectral data and the pre-treated reference values. The created numerical model is output to the outside of the analyzer 23 and is used for actual spectrometry (for example, estimation of the concentration of a predetermined component in a culture solution).

<Evaluation of Numerical Model Created by Numerical Model Creation System 20>

Next, the evaluation of the numerical model created by the numerical model creation system 20 will be described based on examples.

First, creation of a numerical model using a method of the related art will be described.

First, CHO (Chinese Hamster Ovary) cells were cultured eight times. Hereinafter, the eight culture processes will be referred to as A, B, C, D, E, F, G, and H, respectively. In the culture solution used, glucose and L-glutamine were added to a Dulbecco's modified eagle pre-culture liquid medium with low glucose (1.0 [g/1]), and the concentration of glucose was adjusted to be 5.5 to 6.5 [g/l] to perform culture.

The eight (A to H) culture processes were performed using 125 [ml] of a pre-culture liquid medium in an incubator at an air temperature of 37.5° C. in an environment where the concentration ratio of CO₂ to air was 5%.

In four culture processes (A to D) among the eight (A to H) culture processes, five samples were obtained at a frequency of once per day to perform the spectrometry and the reference value measurement. A numerical model was created using spectral data and reference values obtained from each of a plurality of samples.

Specifically, first, a numerical model was created using data (spectral data and reference values) of five samples obtained each of the four culture processes (A, B, C, D). In this case, four numerical models were created.

Next, a numerical model was created using each of all the data (spectral data and reference values) obtained from all the combinations (AB, AC, AD, BC, BD, and CD) of two culture processes among the four culture processes (A to D). In this case, six numerical models were created.

Next, a numerical model was created using each of all the data (spectral data and reference values) obtained from all the combinations (ABC, ABD, ACD, and BCD) of three culture processes among the four culture processes (A to D). In this case, four numerical models were created.

Further, a numerical model was created using each of all the data (spectral data and a reference value) obtained from all the combination (ABCD) of four culture processes among the four culture processes (A to D). In this case, one numerical model was created.

On the other hand, in the pseudo sample creation method according to the first embodiment, a culture solution after completion (eight days after culture) of a single culture process (for example, A) among the four culture processes (A to D) was adopted as the post-culture liquid medium 2, and the pseudo sample 4 was created. As the pre-culture liquid medium 1, a Dulbecco's modified eagle medium with low glucose (1.0 [g/l]) was adopted. As the additive 3, glucose was adopted.

The pre-culture liquid medium 1, the post-culture liquid medium 2, and the additive 3 were mixed with one another in random fractions to prepare 66 types of pseudo samples 4 having different mixing ratios. The 66 types of pseudo samples 4 were sequentially input to the numerical model creation system 20 (FIG. 4) to create a numerical model. As the multi-variable analysis in the analyzer 23, PLS regression was adopted.

Spectral data and reference values were acquired from a culture solution after completion (eight days after culture) of the remaining four culture processes (E to H) among the eight (A to H) culture processes and were used to verify the accuracy of the numerical model. Specifically, the spectral data was used to estimate the concentration of glucose using the numerical model. The reference value was used as a true value of the concentration of glucose.

Next, FIG. 8 is a diagram illustrating an example of a numerical sample created using the pseudo sample creation method according to the first embodiment.

In FIG. 8, the horizontal axis represents the number of culture solutions used for creating a numerical sample. The number of culture solutions used to create a numerical sample in the method of the related art is one to four, and the number of culture solutions used to create a numerical sample in the pseudo sample creation method according to the first embodiment is one. In FIG. 8, the vertical axis represents an error index RMSEP [g/l] between a concentration of glucose estimated using a numerical sample and a true value of the concentration of glucose. As the value of RMSEP decreases, the accuracy of the numerical model increases. When the value of RMSEP is 0.6 [g/l] or lower, the accuracy of the numerical model can be considered to be high.

In FIG. 8, white circles represent values of RMSEP corresponding to the method of the related art, black triangles represent average values of RMSEP corresponding to the method of the related art, and a black square represents a value of RMSEP corresponding to the pseudo sample creation method according to the first embodiment.

It can be seen that in the method of the related art, as the number of culture solutions used to create a numerical model increases, the number of data increases; therefore, the high accuracy of the numerical model can be realized.

On the other hand, it can be seen that, in the pseudo sample creation method according to the first embodiment, the number of culture solutions (post-culture liquid medium 2) used to create a numerical model was one, but 66 types of pseudo samples 4 having different mixing ratios between the pre-culture liquid medium 1 and the additive 3 were created; therefore, a high-accuracy numerical model in which RMSEP is 0.57 [g/l] was able to be created.

The reason why a high-accuracy numerical model can be created using the pseudo sample 4 created by the pseudo sample creation method according to the first embodiment can be described by assuming that a numerical model is configured with two factors including a principal factor and an inhibiting factor.

That is, the principal factor is a factor that determines the true component concentration and, here, corresponds to single spectra of glucose. The inhibiting factor is a factor that provides error to the component concentration determined by the principal factor and, here, corresponds to lactic acid or the like. The pseudo sample 4 created by changing mixing ratios among the pre-culture liquid medium 1, the post-culture liquid medium 2, and the additive 3 succeeds to remove a concentration correlation between the principal factor and the inhibiting factor such that the principal factor and the inhibiting factor can be clearly distinguished from each other. Therefore, it is presumed that a high-accuracy numerical model can be created.

### Pseudo Sample Measurement System 100 According to Third Embodiment of Present Invention

In the pseudo sample creation method according to the first embodiment, the pseudo sample is manually created. However, the pseudo sample may be automatically created.

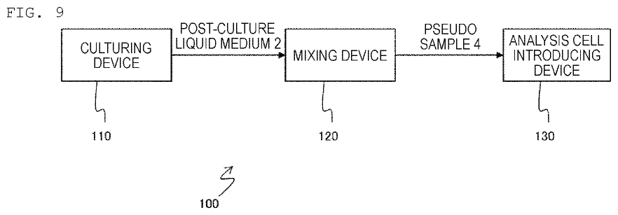

Next, FIG. 9 illustrates a configuration example of a pseudo sample measurement system 100 according to a third embodiment of the present invention.

The pseudo sample measurement system 100 automatically executes a process of creating the pseudo sample 4 and introducing the pseudo sample 4 into the analysis cell without manually executing the process. The pseudo sample measurement system 100 includes a culturing device 110, a mixing device 120, and an analysis cell introducing device 130.

The culturing device 110 creates the post-culture liquid medium 2 and outputs the post-culture liquid medium 2 to the mixing device 120. The mixing device 120 mixes the pre-culture liquid medium 1 and the additive 3 with the post-culture liquid medium 2 output from the culturing device 110 to create the pseudo sample 4. The analysis cell introducing device 130 introduces the pseudo sample 4 created by the mixing device 120 to the analysis cell 201.

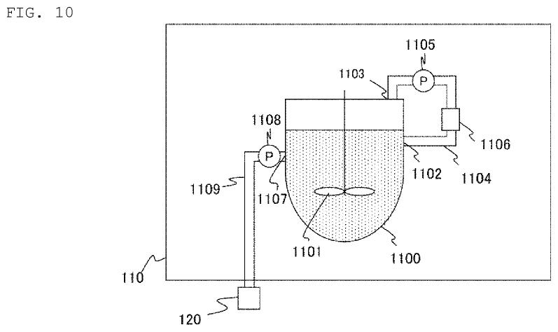

Next, FIG. 10 illustrates a configuration example of the culturing device 110.

The culturing device 110 includes a culture tank 1100, a first pump 1105, an optical analysis cell 1106, and a second pump 1108.

A stirring blade 1101 is provided in the culture tank 1100. In addition, a first outlet 1102 and an inlet 1103 are provided in the culture tank 1100. The first outlet 1102 and the inlet 1103 are connected to each other through a first flow path 1104. On the first flow path 1104, the first pump 1105 and the optical analysis cell 1106 are provided.

Further, a second outlet 1107 is provided in the culture tank 1100. The second outlet 1107 is connected to the mixing device 120 through a second flow path 1109. On the second flow path 1109, the second pump 1108 is provided.

It is desirable that the first flow path 1104 and the second flow path 1109 have high heat resistance, pressure resistance, and mechanical strength, have easiness of cleaning, sterilization, and the like, and are non-invasive to materials in the culture tank 1100. The optical analysis cell 1106 is formed of a material that allows transmission of light, for example, fused silica or an acrylic resin.

In the culturing device 110, in a state where the first outlet 1102 of the culture tank 1100 is filled with the culture solution up to the upstream side and the sample is put into the culture tank 1100, the first outlet 1102 and the inlet 1103 are opened, the second outlet 1107 is closed, and the first pump 1105 is driven. As a result, the culture solution flows out from the first outlet 1102, passes through the first flow path 1104, and returns to the culture tank 1100 through the inlet 1103. By performing optical analysis on the optical analysis cell 1106 on the first flow path 1104, the progress status of culture of the sample in the culture solution can be checked. In the culturing device 110, the optical analysis can be performed on the optical analysis cell 1106 without exposing the culture solution to external air. Therefore, incorporation of contaminants can be prevented during the optical analysis.

When it is verified from the optical analysis that the progress status of culture is desirable, the first outlet 1102 and the inlet 1103 are closed, the first pump 1105 is stopped, the second outlet 1107 is opened, and the second pump 1108 is driven. As a result, the culture solution (post-culture liquid medium 2) flows out from the second outlet 1107, passes through the second flow path 1109, and is output to the mixing device 120.

In the culturing device 110, the culture solution in which the progress status of culture is desirable can be obtained by using one culture tank 1100 without using a plurality of culture tanks 1100.

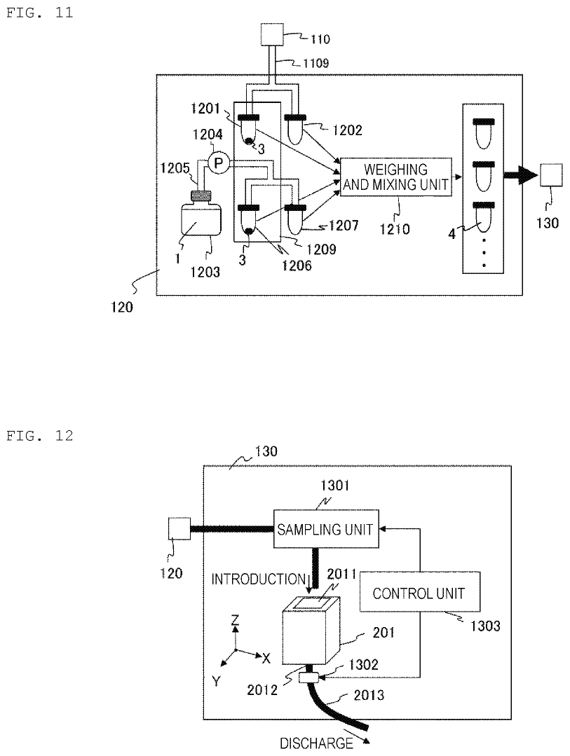

Next, FIG. 11 illustrates a configuration example of the mixing device 120. The mixing device 120 corresponds to the pseudo sample creation apparatus according to the present invention.

The mixing device 120 includes a first container 1201, a second container 1202, a third container 1203, a pump 1204, a flow path 1205, a fourth container 1206, a fifth container 1207, a shaker 1209, and a weighing and mixing unit 1210.

It is desirable that the first container 1201, the second container 1202, the third container 1203, the flow path 1205, the fourth container 1206, and the fifth container 1207 have high heat resistance, pressure resistance, and mechanical strength, have easiness of cleaning, sterilization, and the like, and are non-invasive to materials in the culture tank 1100.

In the first container 1201 and the fourth container 1206, a predetermined amount of the additive 3 is stored in advance. In the third container 1203, the pre-culture liquid medium 1 is stored. The post-culture liquid medium 2 is introduced from the culturing device 110 into the first container 1201 and the second container 1202. The pre-culture liquid medium 1 is introduced from the third container 1203 into the fourth container 1206 and the fifth container 1207.

The shaker 1209 shakes the first container 1201 and the fourth container 1206. The weighing and mixing unit 1210 creates the pseudo sample 4 by weighing, acquiring, and mixing the culture solution (the pre-culture liquid medium 1, the pre-culture liquid medium 1 with which the additive 3 is mixed, the post-culture liquid medium 2, and the post-culture liquid medium 2 with which the additive 3 is mixed) introduced from the first container 1201, the second container 1202, the fourth container 1206, and the fifth container 1207.

In the mixing device 120, the post-culture liquid medium 2 is introduced from the culturing device 110 into the first container 1201 and the second container 1202. In addition, the pump 1204 is driven such that the pre-culture liquid medium 1 stored in the third container 1203 is introduced into the fourth container 1206 and the fifth container 1207 through the flow path 1205.

Next, the shaker 1209 shakes the first container 1201 that stores the post-culture liquid medium 2 and the fourth container 1206 that stores the pre-culture liquid medium 1 to be mixed with the additive 3 stored in advance. Next, the weighing and mixing unit 1210 creates a plurality of pseudo samples 4 having different mixing ratios by weighing, acquiring and mixing the culture solutions from the first container 1201, the second container 1202, the fourth container 1206, and the fifth container 1207.

Next, FIG. 12 illustrates a configuration example of the analysis cell introducing device 130.

The analysis cell introducing device 130 includes a sampling unit 1301, a control valve 1302, and a control unit 1303.

The sampling unit 1301 introduces the pseudo sample 4 input from the mixing device 120 into the analysis cell 201 in accordance with the control from the control unit 1303.

Although not illustrated in the drawing, the analysis cell 201 illustrated in FIG. 12 is held by the holding unit 214 of the spectrometer (FIG. 5), and spectrometry can be performed immediately after the pseudo sample 4 is introduced.

The control valve 1302 is provided in a waste liquid tube 2013 that is connected to a discharge port 2021 provided on the lower surface of the analysis cell 201. The control valve 1302 is, for example, a solenoid valve and is opened in accordance with control from the control unit 1303 such that the pseudo sample 4 stored in the analysis cell 201 is discharged.

It is desirable that the waste liquid tube 2013 has high heat resistance, pressure resistance, and mechanical strength, is deformable, has easiness of cleaning, sterilization, and the like, and is formed of a material such as rubber, silicon, or Tygon.

The control unit 1303 is implemented by, for example, a computer including a CPU, a memory, a communication interface, and a storage. The control unit 1303 controls the sampling unit 1301 and the control valve 1302.

In the analysis cell introducing device 130, first, the control valve 1302 is closed by the control unit 1303. Next, the sampling unit 1301 introduces the pseudo sample 4 input from the mixing device 120 into the analysis cell 201 through the introduction port 2011 in accordance with the control from the control unit 1303. In a state where the pseudo sample 4 is stored in the analysis cell 201, spectrometry or the like is performed. After the analysis ends, the control valve 1302 is opened by the control unit 1303. As a result, the pseudo sample 4 is discharged from the analysis cell 201 through the waste liquid tube 2013. The above-described operation is repeated by the number of the pseudo samples 4.

In the above-described pseudo sample measurement system 100, the series of processes including the creation of the post-culture liquid medium 2, the creation of the pseudo sample 4, the introduction of the pseudo sample 4 into the analysis cell 201, the optical analysis of the pseudo sample 4, and the replacement of the pseudo sample 4 in the analysis cell 201 can be automatically executed without being manually executed.

The present invention is not limited to the embodiments and the modification examples and includes various modification examples. For example, the respective embodiments have been described in detail in order to easily describe the invention, and the present invention is not necessarily to include all the components described above. In addition, a part of the configuration of one embodiment can be replaced with the configuration of another embodiment. Further the configuration of one embodiment can be added to the configuration of another embodiment. In addition, addition, deletion, and replacement of another configuration can be made for a part of the configuration each of the embodiments.

### REFERENCE SIGNS LIST

1: pre-culture liquid medium

2: post-culture liquid medium

3: additive

4: pseudo sample

20: numerical model creation system

21: spectrometer

22: reference value measuring device

23: analyzer

100: pseudo sample measurement system

110: culturing device

120: mixing device

130: analysis cell introducing device

201: analysis cell

211: controller

212: light source unit

213: light receiving unit

214: holding unit

215: fixing tool

231: pre-processing unit

232: calculating unit

233: storage unit

1100: culture tank

1101: stirring blade

1102: first outlet

1103: inlet

1104: first flow path

1105: first pump

1106: optical analysis cell

1107: second outlet

1108: second pump

1109: second flow path

1201: first container

1202: second container

1203: third container

1204: pump

1205: flow path

1206: fourth container

1207: fifth container

1209: shaker

1210: weighing and mixing unit

1301: sampling unit

1302: control valve

1303: control unit

2011: introduction port

2013: waste liquid tube

2021: discharge port

1. A pseudo sample creation method comprising:
mixing a pre-culture liquid medium that is not used to culture a sample, a post-culture liquid medium that is used to culture the sample, and an additive with one another to create a pseudo sample.

2. The pseudo sample creation method according to claim 1,
wherein the additive contains at least one of saccharides, an amino acid, a metabolite, and protein.

3. The pseudo sample creation method according to claim 1,
wherein the pre-culture liquid medium does not contain saccharides and an amino acid or contains a predetermined amount or less of at least one of saccharides and an amino acid.

4. The pseudo sample creation method according to claim 1,
wherein the post-culture liquid medium is a liquid medium in which the sample is not removed or is removed from the culture solution used to culture the sample.

5. The pseudo sample creation method according to claim 1,
wherein a proportion of each of the pre-culture liquid medium and the post-culture liquid medium with respect to the volume of the pseudo sample is 0% to 100%.

6. The pseudo sample creation method according to claim 1, further comprising:
creating a plurality of the pseudo samples having different mixing ratios among the pre-culture liquid medium, the post-culture liquid medium, and the additive.

7. A pseudo sample creation apparatus comprising:
a weighing and mixing unit that creates a pseudo sample by weighing and mixing a pre-culture liquid medium which is not used to culture a sample, a post-culture liquid medium which is used to culture the sample, and an additive with one another.

8. The pseudo sample creation apparatus according to claim 7, further comprising:
a shaker that shakes the pre-culture liquid medium with which the additive is mixed and the post-culture liquid medium with which the additive is mixed.

9. A numerical model creation system comprising:
a pseudo sample creation apparatus that creates a pseudo sample by weighing and mixing a pre-culture liquid medium which is not used to culture a sample, a post-culture liquid medium which is used to culture the sample, and an additive with one another;
a spectrometer that acquires spectral data of the pseudo sample;
a reference value measuring device that measures a reference value of the pseudo sample; and
an analyzer that creates a numerical model representing a correlation between the spectral data and the reference value based on the spectral data and the reference values obtained from a plurality of pseudo samples having different mixing ratios among the pre-culture liquid medium, the post-culture liquid medium, and the additive.

10. The numerical model creation system according to claim 9,
wherein the analyzer creates the numerical model by multi-variable analysis.
